(19) United States
(12) Patent Application Publication
(10) Pub. No.: US 2007/0255224 A1
(43) Pub. Date: Nov. 1, 2007

(54) Facet injection kit

(76) Inventors: James Ahern, Austin, TX (US); Julia Talley, Austin, TX (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/784,767

(22) Filed: Apr. 9, 2007

Related U.S. Application Data
(60) Provisional application No. 60/790,233, filed on Apr. 7, 2006.

(51) Int. Cl. A61M 5/00

(52) U.S. Cl. 604/187

(57) ABSTRACT

One embodiment of the present invention is a facet injection kit for injecting a mixture of anesthesia and steroid into a patient's spinal facet joint. The kit includes antibiotic solution, a basin for holding the antibiotic solution, an antibiotic applicator for applying the antibiotic solution, a local anesthesia, a syringe for holding the local anesthesia and a steroid solution, a needle for drawing the local anesthesia and steroid solution into the syringe, and a needle for injecting the local anesthesia and steroid solution into the patient's spinal facet joint.

### FIELD OF THE INVENTION

The present invention relates generally to administering spinal injections to patients, and more particularly to a facet injection kit and method.

### BACKGROUND OF THE INVENTION

The normal human spine includes thirty-three vertebrae, each vertebra containing a facet joint. Each facet joint is located on the back of the spine, where one vertebra slightly overlaps the adjacent vertebra, guiding and restricting the movement of the spine. As a result of age, injury, disease, or otherwise, the facet joint can become inflamed and become a source of pain. To remedy this pain, physicians may inject a combination of anesthetic and steroid into the one or more facet joints, while employing fluoroscopy techniques.

To provide any kind of spinal injection, a physician requires a variety of equipment such as syringes, needles, basins, and more, along with multiple pharmaceuticals. One of the most common spinal procedures is to inject epidural anesthesia into a patient. This use of epidural anesthesia is well established, and the procedure is commonly performed in hospitals and clinics throughout the world. As such, a number of epidural anesthesia kits are readily available to physicians administering epidural anesthesia. These kits provide all the necessary equipment, supplies, and pharmaceuticals required to perform the procedure, including multiple pre-filled syringes, epidural needles, catheters, saline, and other items required to administer an epidural injection.

Many of the items that are used to administer an epidural injection are also used to administer facet injections. Because of this, and because of the widespread availability of convenient epidural anesthesia kits, physicians often open and use epidural anesthesia kits when performing facet injections. However, although a typical epidural anesthesia kit provides enough equipment, supplies, and pharmaceuticals to perform a single facet injection, the epidural anesthesia kit contains additional items that are not used in the facet injection procedure.

Therefore, when a physician opts for using a readily-available epidural anesthesia kit to perform a facet injection, he increases the time needed to perform the facet injection, creates waste, and ultimately raises the cost of the procedure. For example, the extra, unused items in the epidural anesthesia kit are typically discarded. Also, physicians and nurses must sort through the epidural anesthesia kit to separate the necessary items from the unnecessary items in the kit. This not only wastes physician and staff time, but also underutilizes expensive fluoroscopic equipment.

It would be desirable to provide physicians and medical personnel with a spinal injection kit designed specifically for facet injections. Such a kit would potentially reduce waste, save storage space and costs, decrease procedure preparation time, decrease the amount of fluoroscopy time, all leading to reduced procedure costs and increased convenience to physicians and medical staff.

### SUMMARY OF INVENTION

The invention substantially meets the aforementioned needs of the industry by providing a kit that provides the specific equipment, supplies, and pharmaceuticals needed to administer a facet injection while reducing waste, shortening procedure time, and increasing convenience.

One embodiment of the invention comprises a kit that includes an inner and outer container, a basin, antibiotic solution and applicator, syringe, a large-bore needle, a small-bore needle with stylet, and a sterile sealing drape. The kit may also include items such as a local anesthetic agent, steroid solution, gauze pad, wound dressing, drape cleansing towel, and additional small-bore needles. The invention also includes a method of using the kit to perform a facet injection. The method includes such steps as placing the patient on a fluoroscopy table, opening the kit, cleaning the patient's skin using materials from the kit, imaging the patient's back, filling the syringe with local anesthetic and a steroid solution, inserting needles into a facet joint, and injecting the local anesthetic and steroid solution into the facet joint. The injection procedure may be repeated as needed for multiple facet joints. A related embodiment also includes using the kit to administer other types of spinal joint injections.

### BRIEF DESCRIPTION OF THE DRAWINGS

FIG. 1 depicts a facet injection kit according to one embodiment of the invention.

### DETAILED DESCRIPTION OF THE PREFERRED EMBODIMENTS

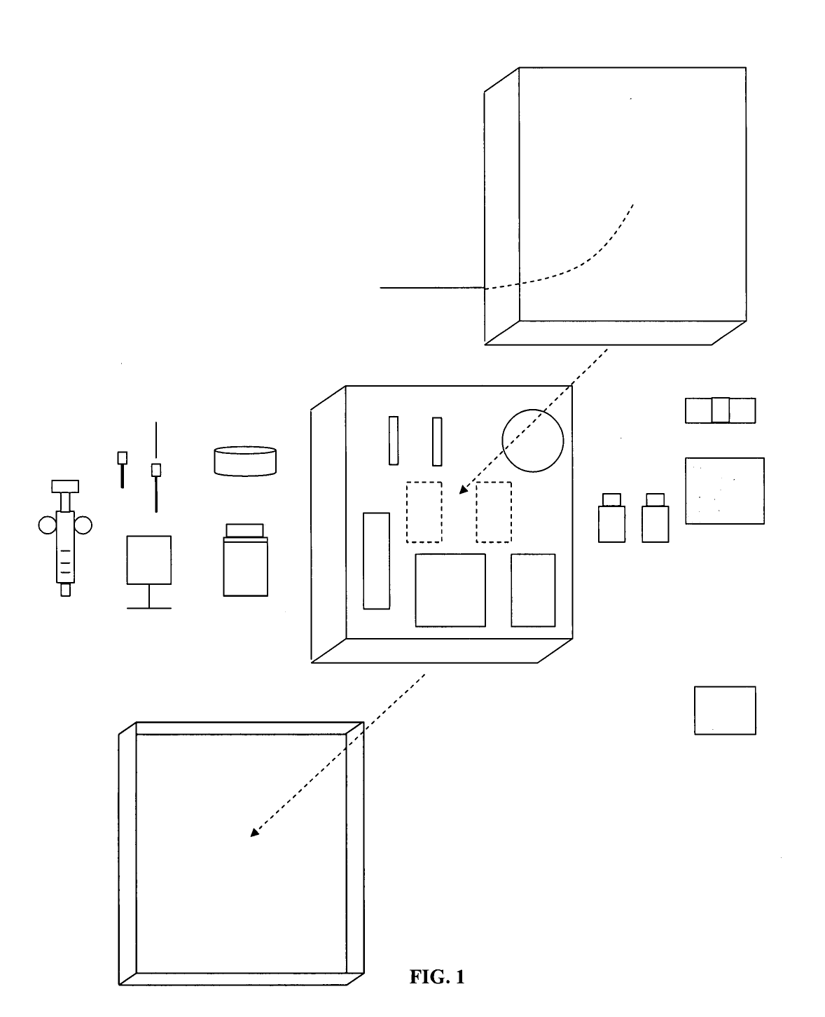

As FIG. 1 illustrates, one preferred embodiment of the present invention, a facet injection kit 100, includes an outer container 101, and an inner container 102 that includes basin 104, antibiotic solution 106, antibiotic applicator 108, a syringe 110, a large-bore needle 112, a small-bore needle 114, a stylet 113 inserted in needle 114, and container drape 118. In an alternate embodiment, the kit may also include one or more of the following: ampule or vile 115 of local anesthetic agent 116, additional needles 114, gauze pad 140, wound dressing 142, drape 144, vile 146 of steroid solution 148, and cleansing towel 150.

Outer container 101 houses inner container 102 and its contents and is designed such that it can be sterilized and opened without comprising its sterility. Inner container 102 includes a number of compartments 120 through 125 for retaining antibiotic applicator 108, syringe 110, first needle 112, second needle 114 with stylet 113. In some embodiments, inner container 102 includes additional compartments 126 and 127 for retaining optional vile 115 of local anesthetic agent 116 and vile 146 of steroid solution 148. In one preferred embodiment, outer container 101 is a six-sided container which is placed over inner container 102 such that upper surface 152 of inner container 102 is adjacent to surface 154 of outer container 101, and sealing drape 118 is adhesively attached to bottom surface 156 of inner container 102 and outer container 101.

In an alternate embodiment, inner container 102 is placed into outer container 101 such that bottom surface 158 of inner container 102 is adjacent to surface 154 of outer container 101. Sealing drape 118 is adhesively attached to upper surface 152 of inner container 102 and outer container 101.

In the preferred embodiment, antibiotic applicator 108 is sterile, and comprised of a soft, sponge-like material, preferably with a handle 109, and is further able to absorb liquid, and to release liquid onto the patient's skin. Syringe 110 is sterile and capable of holding at least 2 cc of fluid, and preferably includes, a plunger 130, graduation markings 132 in at least 0.25 cc increments, a luer lock fitting 134, a thumb ring 136 and a finger ring 138. Needle 112 is less than 2 inches long, and has a bore size of 20 gauge or larger, and local anesthetic agent 116 is Xylocaine®.

One method of utilizing facet injection kit 100 to administer a facet injection starts with the patient being placed on a fluoroscopy table. The patient is then instructed to roll up onto their side, at about a 45 degree angle. Next, the physician or staff helps the patient into the proper position and removes the patient's clothing to expose the treatment area.

Container drape 118 covering outer container 101 is then peeled back in a sterile manner, followed by the patient's skin being cleaned with antibiotic solution 106, in a normal sterile manner, using antibiotic applicator 108. Next, an image using a fluoroscopic imaging machine is taken of the patient's back to locate the facet joint to be injected.

After the facet joint is located, the patient is prepared per the steps above. The physician puts on sterile gloves of the proper size for the physician's hands and attaches needle 112 to syringe 110. The physician pulls back on plunger 130 of syringe 110 allowing air into syringe 110 and inserts needle 112 into vile 115 of local anesthetic agent 116. The physician then depresses plunger 130 of syringe 108 causing air within syringe 110 to flow into vile 115 of local anesthetic agent 116. Anesthetic agent 116 will then flow from an area of greater pressure to an area of lower pressure, from the ampule or vile 115 of local anesthetic agent 116 into syringe 110. The physician then determines when the proper amount of local anesthetic agent 116 is in syringe 110.

Next, the physician depresses plunger 130, causing a very small amount, in the range of about less than 0.1 cc, of local anesthetic agent 116 to return to vile 115. The physician then removes needle 112 from vile 115 of local anesthetic agent 116, and assures that the proper amount of local anesthetic agent 116 is in syringe 110.

After these steps, the physician inserts needle 112 into a vile 146 containing steroid solution 148. In one embodiment, the steroid solution 148 is celestone soluspan. The physician pulls back plunger 130 of syringe 110, causing the steroid solution 148 to flow from vile 146 into syringe 108. The physician stops withdrawing plunger 130 when the desired amount of steroid solution 148 is in syringe 110.

Next, the physician removes needle 112 from syringe 110. Small bore needle 114, in one embodiment having a proper length small bore of 22 gauge, is opened in a sterile manner and deposited onto kit 100. Needle 114 is placed into the patient's back and positioned properly by the physician using fluoroscopic assistance. Alternatively, additional needles 114 may be placed into the patient's back, depending on the desired treatment.

Stylet 113 of needle 114 is removed from needle 114 and placed on sterile surface 152 in inner container 102 by the physician. If the procedure requires the use of a additional needles 114, the doctor must be careful to remember which stylet 113 was inside which needle 114.

The physician connects syringe 110 containing local anesthetic 116 and steroid solution 148 onto needle 114, which is placed into the patient's facet joint capsule, via luer lock fitting 134. Then the physician depresses plunger 130 of syringe 110 causing local anesthetic 116 and steroid solution to be expelled from syringe 110 through needle 114 and into the facet joint capsule. After the physician has expelled the proper amount of steroid solution, the physician stops depressing plunger 130, removes syringe 110 and needle 114 from the patient and disconnects needle 114 from syringe 110 via luer lock fitting 134. In some situations, the physician may choose to make multiple injections, inserting additional needles 114 at other potentially pathologic facet joints and repeating the process.

After removing needle 114, the physician or assistant may use a cleansing towel 150 to wipe the antibiotic solution 106 from the patient's skin, and apply a wound dressing 142 if hemostasis cannot be achieved. The patient can then sit upright and get off the fluoroscopic table.

The invention therefore provides a kit that conveniently allows physicians and medical staff to administer facet injections in an efficient, cost-effective manner. Furthermore, although the example embodiment described above refers to the use of the kit for administering facet injections, the kit may also be used for administering other types of spinal joint injections.

The invention may be embodied in other specific forms without departing from the spirit of the essential attributes thereof. Therefore, the illustrated embodiments should be considered in all respects as illustrative and not restrictive.

1. A facet injection kit for injecting a mixture of anesthesia and steroid into a patient's spinal facet joint, the kit comprising:
an antibiotic solution;
a steroid solution;
a local anesthesia;
a basin for holding the antibiotic solution;
an antibiotic applicator for applying the antibiotic solution;
a syringe for holding the local anesthesia and the steroid solution;
a first needle for drawing the local anesthesia and steroid solution into the syringe; and
a second needle for injecting the local anesthesia and steroid solution into the patient's spinal facet joint.

2. The kit of claim 1 further including at least 1 additional needle.

3. The kit of claim 1 further including at least one stylet.

4. A facet injection kit for injecting a mixture of anesthesia and steroid into a patient's spinal facet joint, the kit comprising:
an outer container adapted to contain an inner container such that the inner container remains sterile;
the inner container including a plurality of compartments;
at least one of the plurality of compartments is adapted to retain an antibiotic applicator;
at least one of the plurality of compartments is adapted to retain a syringe;
at least one of the plurality of compartments is adapted to retain a first needle;
at least one of the plurality of compartments is adapted to retain a second needle;
an antibiotic solution;
a steroid solution;
a local anesthesia;
a basin for holding the antibiotic solution;
an antibiotic applicator for applying the antibiotic solution;
a syringe for holding the local anesthesia and the steroid solution;
the first needle adapted for drawing the local anesthesia and steroid solution into the syringe; and
the second needle adapted for injecting the local anesthesia and steroid solution into the patient's spinal facet joint.

5. The kit of claim 4 wherein at least one of the plurality of compartments is adapted to retain a vial of local anesthetic.

6. The kit of claim 5 wherein at least one of the plurality of compartments is adapted to retain a vial of steroid solution.

7. The kit of claim 5 wherein the second needle includes a stylet.
